(19) United States
(12) Patent Application Publication
(10) Pub. No.: US 2024/0241070 A1
(43) Pub. Date: Jul. 18, 2024

(54) Ultra-Sensitive Tin Oxide Sensor for Room Temperature Detection of Oxidizing and Reducing Gases

(71) Applicant: University of Puerto Rico, San Juan, PR (US)

(72) Inventors: Wilfredo Otaño-Rivera, Cayey, PR (US); Adrian Camacho-Berrios, Cayey, PR (US)

(73) Assignee: University of Puerto Rico, San Juan, PR (US)

(21) Appl. No.: 18/415,604

(22) Filed: Jan. 17, 2024

Related U.S. Application Data
(60) Provisional application No. 63/439,367, filed on Jan. 17, 2023.

(51) Int. Cl. G01N 27/12; G01N 33/00

(52) U.S. Cl. CPC G01N 27/125; G01N 33/0027

(57) ABSTRACT

A gas sensor and a method of fabrication wherein the sensor includes a polymer fiber substrate and a metal oxide nanoshell. The nanoshell is deposited on the substrate using magnetron sputtering deposition. The sensor can be designed to detect specific gases, such as NO2 or NH3, by using different metal oxides such as tin oxide or zinc oxide. The polymer fiber substrate can be a cellulose acetate substrate prepared by electrospinning. The method for making the sensor involves electrospinning polymeric fibers, depositing a metal oxide on the fibers to produce a nanoshell, and heating the fibers and oxide in air to remove the fibers. The gas sensor uses the nanoshell for the transduction process.

### CROSS-REFERENCE TO RELATED APPLICATIONS

This application is a Non-Provisional Utility Patent application claiming priority to U.S. Provisional patent Application Ser. No. 63/439,367, filed on Jan. 17, 2023, which is incorporated by reference herein in its entirety.

### BACKGROUND OF THE INVENTION

### 1) Field of the Invention

The invention relates in general to the field of gas detection sensors, and more particularly, to fabrication of the gas detection sensors using metal oxides.

### 2) Description of Related Art

Gas sensors are devices that detect and identify different types of gases in the environment. These sensors are used in a variety of applications, including environmental monitoring, industrial process control, and safety systems. The detection of gases is typically achieved through the interaction of the gas with a sensing material, which changes its properties in response to the gas. The change in properties can then be measured and used to identify the presence and concentration of the gas. One common type of gas sensor is based on metal oxides. Metal oxide gas sensors operate by changing their electrical resistance in response to the gas being detected. The metal oxide material is typically deposited on a substrate, and the gas interacts with the surface of the metal oxide, causing a change in the electrical resistance of the material. This change in resistance can then be measured and used to determine the presence and concentration of the gas.

Various methods are used to deposit the metal oxide material on the substrate. One such method is magnetron sputtering deposition, a physical vapor deposition technique that uses a magnetron to generate a plasma of the metal oxide material. The plasma is then used to deposit the metal oxide material onto the substrate. The substrate used in gas sensors is often a polymer fiber substrate. Polymer fibers offer several advantages as substrates for gas sensors, including high surface area, flexibility, and ease of fabrication. One common method of fabricating polymer fiber substrates is electrospinning, a process that uses an electric field to draw out and deposit fibers from a polymer solution.

Specific types of gases that are commonly detected by gas sensors include nitrogen dioxide (NO₂) and ammonia (NH₃). These gases are of particular interest due to their impact on air quality and human health. Nitrogen dioxide is a major air pollutant that contributes to the formation of smog and acid rain, while ammonia is a common industrial chemical that can be harmful if inhaled in large amounts.

These and other objects, features, and advantages of the present invention will become more readily apparent from the attached drawings and the detailed description of the preferred embodiments, which follow.

### SUMMARY OF THE INVENTION

Bearing in mind the problems and deficiencies of the prior art, it is therefore an object of the present invention to provide a gas sensor for detecting harmful gases.

It is another object of the present invention to provide a gas sensor which uses metal oxides for the fabrication of the gas sensor.

It is yet another object of the present invention to provide a method for making a gas sensor which includes electrospinning a polymeric substrate and using magnetron sputtering for depositing a nanostructure on the polymeric substrate.

Still other objects and advantages of the invention will in part be obvious and will in part be apparent from the specification.

The above and other objects, which will be apparent to those skilled in the art, are achieved in the present invention which is directed to the development of zinc and tin oxides nanoshells as active elements for the detection of important gases that distress the environment. The invention includes sensors having a nanostructure of zinc oxide or tin oxides deposited by magnetron sputtering deposition. Silver nanoparticles provides another way to detect specific gas species in high humidity environments.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1A shows a microscope slide with cellulose acetate prepared by electrospinning;

FIG. 1B shows the cellulose acetate on a microscope slide after ZnO deposition and heating;

FIG. 1C shows a micrograph of typical nanoshells as observed in the scanning electron microscope (SEM);

FIG. 2 is Raman spectra of ZnO nanoshells after heating in air. The samples were prepared at two different gas compositions and different deposition times. Two characteristic peaks corresponding to the excitation in the oxygen and zinc sub-lattices are observed;

FIG. 3 is an XRD;

FIG. 4A shows the morphology of the tin semispherical particles deposited on glass, corresponding to a substrate temperatures of 100° C.;

FIG. 4B shows the morphology of the tin semispherical particles deposited on glass, corresponding to a substrate temperatures of 250° C.;

FIG. 5 is SERS signal of Rhodamine 6G for different silver films on glass substrates.

The inset shows the molecule signal on the bare glass (blue line). The spectra in the inset are the signals with the lowest amplification.

FIG. 6 is a chart showing sensitivity of ZnO thin film prepared at 50:50 0₂:Ar;

FIG. 7 is a table of responses;

FIG. 8 is a chart showing morphology of a TONs (tin oxide nanoparticles) structure;

FIG. 9 is a chart showing morphology of another TONs structure;

FIG. 10 shows sensor response;

FIG. 11 is a chart showing a tin oxide nanoparticle plot of current versus time for two samples;

FIG. 12 is a chart showing relative resistance (R/Rb) of tin oxide sample for three different NO₂ concentrations;

FIG. 13 is a chart showing XRD of Glass and SiOx with a deposition time of 30 seconds and deposition temperatures before thermal oxidation;

FIGS. 14A-14F show SEM images of the Sn nanoparticles at a 60, 30 and 15 second deposition duration at 200° C., using SiOx (top photos) and Glass (bottom photos);

FIGS. 15A-15F show SEM images of the SnO₂ nanoparticles at a 60, 30 and 15 second deposition duration at 200° C., using SiOx (top photos) and Glass (bottom photos);

FIGS. 16A-16F show SEM images of the Sn nanoparticles at a 30, 15 and 10 second deposition duration at 150° C., using SiOx (top photos) and Glass (bottom photos);

FIGS. 17A-17F show SEM images of the SnO₂ nanoparticles at a 30, 15 and 10 second deposition duration at 150° C., using SiOx (top photos) and Glass (bottom photos);

FIGS. 18A-18D show SEM images of the Sn nanoparticles at a 15 and 10 second deposition duration at 100° C., using SiOx (top photos) and Glass (bottom photos);

FIGS. 19A-19D show SEM images of the SnO₂ nanoparticles at a 15 and 10 second deposition duration at 100° C., using SiOx (top photos) and Glass (bottom photos);

FIGS. 20A-20F show SEM images of the Sn nanoparticles at a 30, 15 and 10 second deposition duration at room temperature, using SiOx (top photos) and Glass (bottom photos); and

FIGS. 21A-21F show SEM images of the SnO₂ nanoparticles at a 30, 15 and 10 second deposition duration at room temperature, using SiOx (top photos) and Glass (bottom photos).

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or”, also referred to as “non-exclusive or” unless otherwise indicated. Moreover, the use of the term “including,” as well as other forms, such as “includes” and “included”, should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C”. An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

Prior to a discussion of the preferred embodiment of the invention, it should be understood that while the features and advantages of the invention are illustrated in terms of a gas sensor. The gas sensor comprises a polymer fiber substrate and a metal oxide nanoshell. The polymer fiber substrate serves as the base upon which the metal oxide nanoshell is disposed. In some cases, the polymer fiber substrate may be a cellulose acetate substrate. However, it is to be understood that other types of polymer fiber substrates may also be used in the construction of the gas sensor.

The metal oxide nanoshell is deposited on the polymer fiber substrate. This deposition can be achieved through a process known as magnetron sputtering deposition. The metal oxide used in the nanoshell can vary. In some cases, the metal oxide may be tin oxide. In other cases, the metal oxide may be zinc oxide. However, it is to be understood that other types of metal oxides may also be used in the construction of the nanoshell.

The combination of the polymer fiber substrate and the metal oxide nanoshell forms the basic structure of the gas sensor. The specific composition of the polymer fiber substrate and the metal oxide nanoshell can be varied to tailor the gas sensor for detecting specific types of gases. For instance, a gas sensor with a cellulose acetate substrate and a tin oxide nanoshell may be used for detecting NO2, while a gas sensor with a cellulose acetate substrate and a zinc oxide nanoshell may be used for detecting NH3.

The gas sensor is not limited to the specific compositions mentioned. The polymer fiber substrate and the metal oxide nanoshell can be composed of different materials, depending on the specific requirements of the gas sensor. The choice of materials for the polymer fiber substrate and the metal oxide nanoshell can be made based on factors such as the type of gas to be detected, the operating conditions of the gas sensor, and the desired sensitivity and selectivity of the gas sensor. The preparation of the polymer fiber substrate is a process that involves the use of electrospinning. Electrospinning is a technique that involves the use of an electric field to draw charged threads of polymer solutions or polymer melts up to fiber diameters in the order of some hundred nanometers. In some cases, the polymer fiber substrate may be prepared by electrospinning a cellulose acetate solution. The electrospun fibers are collected on a rotating drum or a stationary collector to form a non-woven mat of the polymer fiber substrate. While electrospinning is one method of preparing the polymer fiber substrate, other methods may also be used. The choice of method for preparing the polymer fiber substrate can be based on factors such as the desired properties of the substrate, the type of polymer used, and the specific requirements of the gas sensor. For instance, other methods of fiber production such as melt blowing or solution blowing could also be used to prepare the polymer fiber substrate. Regardless of the method used, the resulting polymer fiber substrate serves as the base upon which the metal oxide nanoshell is deposited to form the gas sensor.

Magnetron sputtering deposition is a method that involves the use of a magnetron sputtering source to deposit a thin film of metal oxide on the polymer fiber substrate. The magnetron sputtering source generates a plasma of the metal oxide, which is then directed towards the polymer fiber substrate. The metal oxide particles in the plasma collide with the polymer fiber substrate and adhere to it, forming a nanoshell of metal oxide on the substrate.

Magnetron sputtering deposition may be used for the fabrication of the gas sensor. and offers several advantages, such as high deposition rates, good adhesion, and the ability to deposit a wide range of materials. Furthermore, magnetron sputtering deposition allows for the control of the thickness and uniformity of the metal oxide nanoshell, which can be tailored to the specific requirements of the gas sensor. While magnetron sputtering deposition is one method of depositing the metal oxide nanoshell, other methods may also be used. The choice of method for depositing the metal oxide nanoshell can be based on factors such as the desired properties of the nanoshell, the type of metal oxide used, and the specific requirements of the gas sensor. For instance, other methods of deposition such as chemical vapor deposition or atomic layer deposition could also be used to deposit the metal oxide nanoshell. Regardless of the method used, the resulting metal oxide nanoshell serves as a transduction element in the gas sensor, enabling the detection of specific gases.

Tin oxide may be used as the metal oxide in the nanoshell. When tin oxide is exposed to a gas, it can interact with the gas molecules, leading to changes in its electrical resistance. These changes in resistance can be measured and used to detect the presence of the gas. When tin oxide is used in the nanoshell of the gas sensor, it can provide the sensor with high sensitivity and selectivity towards specific gases. For instance, a gas sensor with a tin oxide nanoshell may be particularly effective in detecting NO2. The tin oxide nanoshell can interact with NO2 molecules, leading to measurable changes in the electrical resistance of the nanoshell. These changes can be used to detect the presence and concentration of NO2 in the environment. The use of tin oxide in the nanoshell is not limited to the detection of NO2. The tin oxide nanoshell can be tailored to detect other types of gases based on the specific requirements of the gas sensor. The choice of tin oxide as the metal oxide in the nanoshell can be made based on factors such as the type of gas to be detected, the operating conditions of the gas sensor, and the desired sensitivity and selectivity of the gas sensor. While tin oxide is one type of metal oxide that can be used in the nanoshell, it is to be understood that other types of metal oxides may also be used. The choice of metal oxide for the nanoshell can be based on factors such as the desired properties of the nanoshell, the type of gas to be detected, and the specific requirements of the gas sensor. Regardless of the type of metal oxide used, the resulting nanoshell serves as a transduction element in the gas sensor, enabling the detection of specific gases. The functionality of the gas sensor is not limited to the detection of NO2. The gas sensor can be tailored to detect different types of gases based on the specific requirements of the sensor. The choice of metal oxide for the nanoshell, the composition of the polymer fiber substrate, and the fabrication process can all be adjusted to optimize the gas sensor for detecting specific types of gases. For instance, a gas sensor with a zinc oxide nanoshell may be used for detecting NH3, while a gas sensor with a tin oxide nanoshell may be used for detecting CO2. The specific functionality of the gas sensor can be determined based on factors such as the type of gas to be detected, the operating conditions of the sensor, and the desired sensitivity and selectivity of the sensor.

The polymer fiber substrate may be a cellulose acetate substrate. Cellulose acetate is a type of polymer that can be used to form the substrate for the gas sensor. The use of cellulose acetate as the polymer fiber substrate can be particularly advantageous when tin oxide is used as the metal oxide in the nanoshell. Cellulose acetate has properties that make it suitable for use as a substrate in a gas sensor. For instance, it has good mechanical strength, which can provide the gas sensor with structural stability. Furthermore, cellulose acetate has good thermal stability, which can allow the gas sensor to operate under a wide range of temperatures. The cellulose acetate substrate can be prepared by electrospinning a cellulose acetate solution. The electrospun cellulose acetate fibers are collected on a rotating drum or a stationary collector to form a non-woven mat of the cellulose acetate substrate. The resulting cellulose acetate substrate serves as the base upon which the tin oxide nanoshell is deposited to form the gas sensor. When tin oxide is used as the metal oxide in the nanoshell, the cellulose acetate substrate can provide a suitable base for the deposition of the tin oxide. The tin oxide can adhere well to the cellulose acetate substrate, forming a stable and uniform nanoshell on the substrate. The combination of the cellulose acetate substrate and the tin oxide nanoshell can provide the gas sensor with high sensitivity and selectivity towards specific gases, such as NO2. The use of cellulose acetate as the polymer fiber substrate is not limited to cases where tin oxide is used as the metal oxide. The cellulose acetate substrate can be used with other types of metal oxides in the nanoshell, depending on the specific requirements of the gas sensor. The choice of cellulose acetate as the polymer fiber substrate can be made based on factors such as the type of gas to be detected, the operating conditions of the gas sensor, and the desired sensitivity and selectivity of the gas sensor.

The metal oxide used in the nanoshell may be zinc oxide. Zinc oxide, when used as the metal oxide in the nanoshell, plays a pivotal role in the gas sensor. Zinc oxide is a semiconductor material that has been widely used in gas sensing applications due to its excellent gas sensing properties. When zinc oxide is exposed to a gas, it can interact with the gas molecules, leading to changes in its electrical resistance. These changes in resistance can be measured and used to detect the presence of the gas. When zinc oxide is used in the nanoshell of the gas sensor, it can provide the sensor with high sensitivity and selectivity towards specific gases. For instance, a gas sensor with a zinc oxide nanoshell may be particularly effective in detecting NH3. The zinc oxide nanoshell can interact with NH3 molecules, leading to measurable changes in the electrical resistance of the nanoshell. These changes can be used to detect the presence and concentration of NH3 in the environment. The use of zinc oxide in the nanoshell is not limited to the detection of NH3. The zinc oxide nanoshell can be tailored to detect other types of gases based on the specific requirements of the gas sensor. The choice of zinc oxide as the metal oxide in the nanoshell can be made based on factors such as the type of gas to be detected, the operating conditions of the gas sensor, and the desired sensitivity and selectivity of the gas sensor. The functionality of the gas sensor is not limited to the detection of NH3. The gas sensor can be tailored to detect different types of gases based on the specific requirements of the sensor. The choice of metal oxide for the nanoshell, the composition of the polymer fiber substrate, and the fabrication process can all be adjusted to optimize the gas sensor for detecting specific types of gases. For instance, a gas sensor with a tin oxide nanoshell may be used for detecting CO2, while a gas sensor with a zinc oxide nanoshell may be used for detecting NH3. The specific functionality of the gas sensor can be determined based on factors such as the type of gas to be detected, the operating conditions of the sensor, and the desired sensitivity and selectivity of the sensor.

The fabrication of the gas sensor involves a series of steps, starting with the preparation of the polymer fiber substrate. This is achieved through a process known as electrospinning. Electrospinning involves the use of an electric field to draw charged threads of polymer solutions or polymer melts up to fiber diameters in the order of some hundred nanometers. The electrospun fibers are collected on a rotating drum or a stationary collector to form a non-woven mat of the polymer fiber substrate. This substrate serves as the base upon which the metal oxide nanoshell is deposited. The next step in the fabrication process involves the deposition of a metal oxide on the polymeric fibers. This deposition process results in the formation of a metal oxide nanoshell on the polymeric fibers. The deposition can be achieved through a process known as magnetron sputtering deposition. The magnetron sputtering source generates a plasma of the metal oxide, which is then directed towards the polymer fiber substrate. The metal oxide particles in the plasma collide with the polymer fiber substrate and adhere to it, forming a nanoshell of metal oxide on the substrate. Following the deposition of the metal oxide nanoshell, the polymer fibers and metal oxide are heated in air. This heating process serves to remove the polymeric fibers, leaving behind the metal oxide nanoshell. The heating process can be tailored based on factors such as the type of polymer used, the type of metal oxide used, and the specific requirements of the gas sensor. The resulting gas sensor comprises a metal oxide nanoshell on a polymer fiber substrate, with the nanoshell serving as a transduction element for the detection of specific gases.

The described method for making the gas sensor is not limited to the specific steps and materials mentioned. The choice of polymer for the substrate, the type of metal oxide for the nanoshell, and the specific processes used for electrospinning and deposition can be varied based on the specific requirements of the gas sensor. The described method provides a general framework for the fabrication of a gas sensor, with the specific details being adjustable based on the desired properties of the sensor.

The nanoshell plays a role in the transduction process of the gas sensor. The transduction process is the mechanism by which the gas sensor detects the presence of specific gases. This process involves the interaction between the gas molecules and the metal oxide nanoshell. When the gas molecules come into contact with the nanoshell, they interact with the metal oxide, leading to changes in the electrical resistance of the nanoshell. These changes in resistance can be measured and used to detect the presence and concentration of the gas.

While the disclosed gas sensor utilizes magnetron sputtering deposition for the deposition of the metal oxide nanoshell, other methods of deposition may also be employed. The choice of deposition method can be influenced by factors such as the desired properties of the nanoshell, the type of metal oxide used, and the specific requirements of the gas sensor. For instance, other methods of deposition such as chemical vapor deposition or atomic layer deposition could also be used to deposit the metal oxide nanoshell. Each of these methods has its own advantages and can be selected based on the specific requirements of the gas sensor.

Similarly, while the polymer fiber substrate is prepared by electrospinning in the disclosed gas sensor, other methods of preparing the polymer fiber substrate may also be used. The choice of method for preparing the polymer fiber substrate can be based on factors such as the desired properties of the substrate, the type of polymer used, and the specific requirements of the gas sensor. For instance, other methods of fiber production such as melt blowing or solution blowing could also be used to prepare the polymer fiber substrate. Each of these methods has its own advantages and can be selected based on the specific requirements of the gas sensor. Regardless of the method used for depositing the metal oxide nanoshell or preparing the polymer fiber substrate, the resulting gas sensor comprises a polymer fiber substrate and a metal oxide nanoshell. The nanoshell serves as a transduction element in the gas sensor, enabling the detection of specific gases. The specific composition of the polymer fiber substrate and the metal oxide nanoshell can be varied to tailor the gas sensor for detecting specific types of gases.

The operational parameters of the gas sensor can vary based on several factors. These factors include, but are not limited to, the type of gas to be detected, the operating conditions of the sensor, and the desired sensitivity and selectivity of the sensor. The operational parameters of the gas sensor can be adjusted to optimize the sensor for detecting specific types of gases. For instance, the operating temperature of the gas sensor can be an influential parameter. The operating temperature can affect the sensitivity and selectivity of the gas sensor. In some cases, the gas sensor may operate at room temperature. In other cases, the gas sensor may operate at elevated temperatures. The choice of operating temperature can be made based on factors such as the type of gas to be detected, the type of metal oxide used in the nanoshell, and the specific requirements of the gas sensor.

The concentration of the gas to be detected can also be an operational parameter. The gas sensor can be designed to detect gases at low concentrations, high concentrations, or a range of concentrations. The choice of concentration range can be made based on factors such as the type of gas to be detected, the operating conditions of the sensor, and the desired sensitivity and selectivity of the sensor.

The response time of the gas sensor can be another operational parameter. The response time refers to the time it takes for the gas sensor to detect the presence of a gas once it has been exposed to the gas. The response time can be influenced by factors such as the type of gas to be detected, the type of metal oxide used in the nanoshell, and the specific requirements of the gas sensor. The operational parameters of the gas sensor are not limited to the ones mentioned. Other operational parameters such as the operating pressure, the humidity level, and the flow rate of the gas can also be considered. The choice of operational parameters can be made based on the specific requirements of the gas sensor, with the aim of optimizing the sensor for detecting specific types of gases.

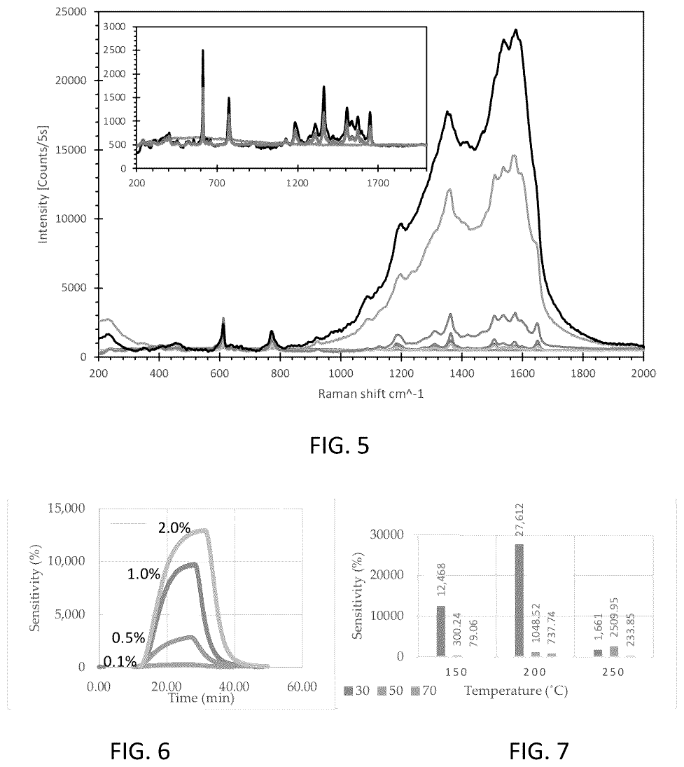

In one embodiment of the present invention, ZnO was deposited simultaneously on interdigital transducers (IDTs) as thin films or as nanoshells, using a sacrificial polymer scaffold, via de pulsed magnetron sputtering. IDTs primary function is to convert electric signals to surface acoustic waves (SAW) by generating periodically distributed mechanical forces via piezoelectric effect (an input transducer). The oxygen/argon ratio in the sputtering gas and the sputtering time were controlled to produce 30 nm thick structures that were tested as transducing material for hydrogen gas. X-ray diffraction shows that the thin films are textured with (002) preferential orientation, while no peaks are observed for the nanoshells due to their semicircular distribution of orientations. The morphology of the surface of the samples, studied by scanning electron microscopy, changes from smooth to grain-like according to the deposition conditions. FIGS. 5A and 5B correspond to the sensitivity, defined as S(%)=ΔR/R×100, of the IDTs with the ZnO thin films. The samples were tested in a home-made system allowing to measure down to 0.1% of hydrogen in argon. Very high sensitivities were obtained for the films prepared at oxygen deficient conditions where a larger number of oxygen vacancies are present. The increase in current is consistent with an n-type semiconductor where hydrogen atoms produce the desorption of oxygen and OH, resulting in the incorporation of electrons to the material. On the other hand, the nanoshell response (not shown) was significantly smaller, possibly creating problems with the contacts on the gold pads of the IDTs.

### Synthesis of Silver Nanostructures for Surface Enhanced Raman Spectroscopy

Silver discontinuous structures are key players in surface enhanced Raman spectroscopy (SERS) where specific molecules are detected at nano-molar concentrations. The use of SERS could be instrumental for the detection of specific gases of interest in the wetland by using molecules that will not passivate in the presence of water vapor. In this first stage, the formation of silver nanostructures by magnetron sputtering deposition is explored.

### Silver Nanoparticles for SERS Gas Sensing

Significant Raman enhancement of Raman molecule Rhodamine 6G (R6G) has been made using silver nanoparticles synthesized using plasma vapor deposition (PVD). Altering plasma condition in PVD could change the morphology of the silver nanoparticles, which in turn varies the degree of Raman enhancement obtained from R6G. Nano-island morphology of the silver nanoparticles obtained the highest Raman enhancement.

The gas sensors may have certain aspects of selectivity for different gases and selectivity based on exposure to humid environments.

The computational approach provides a simulated system with silver nanoparticles attached to different molecules such as thiophenol. Calculation of the necessary components to simulate a Raman spectrum may be difficult as parameters for calculating and obtaining polarizability of the molecules are difficult and time consuming. An alternate approach to simulating a Raman spectrum directly is to first simulate an Infrared spectrum and then transform the data to allow simulation of a Raman spectrum. Both optical spectroscopy techniques rely on vibrational and rotational modes of the molecules. Dipole energy potentials were calculated for every atom in the system using NWChem to obtain the parameters needed to simulate the Infrared spectrum. Results have shown difficulties in obtaining converging energy minimums which require use of a different computational software such as VASP.

Sputtering is directed to the development of zinc and tin oxides nanoshells as active elements for the detection of important gases that distress the environment. A description follows of two present efforts of creating novel nanostructures of zinc and tin oxides by magnetron sputtering deposition. Magnetron sputtering is used for the deposition of ZnO. An rf power supply or pulsed dc power supply may be used.

ZnO samples were deposited on interdigital electrodes varying plasma deposition parameters. The rf power generator may use a 13.5 MHz frequency which is high when compared with the kilohertz frequency used in the dc pulsed power supply, changing the plasma deposition conditions. These changes result in a dramatic reduction in the sputtering rate of the zinc target and, consequently, in the composition of the deposited films. In particular, the amount of point defects is affected resulting in a different gas response.

Synthesis of tin oxide nanostructures for sensing of biologically important gases - Tin nanoparticles are deposited on interdigitated electrodes (IDEs) by magnetron sputtering. Two IDEs are deposited simultaneously and one of them receives a heat post-treatment. Both samples are tested for their response to 50 parts per million of N0₂ in N₂. Both samples have similar response, a reduction in current when the 02 is introduced, but the post-treated sample shows a significant increase in current. Both samples show sensitivities of over 600%. In general, the samples with the heat post-treatment show better response and a significant increase in current magnitude, a promising result for gas sensing.

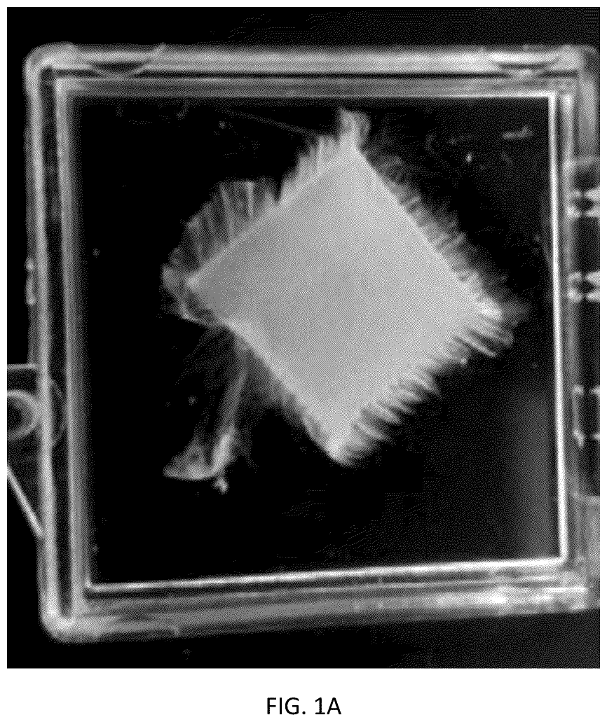

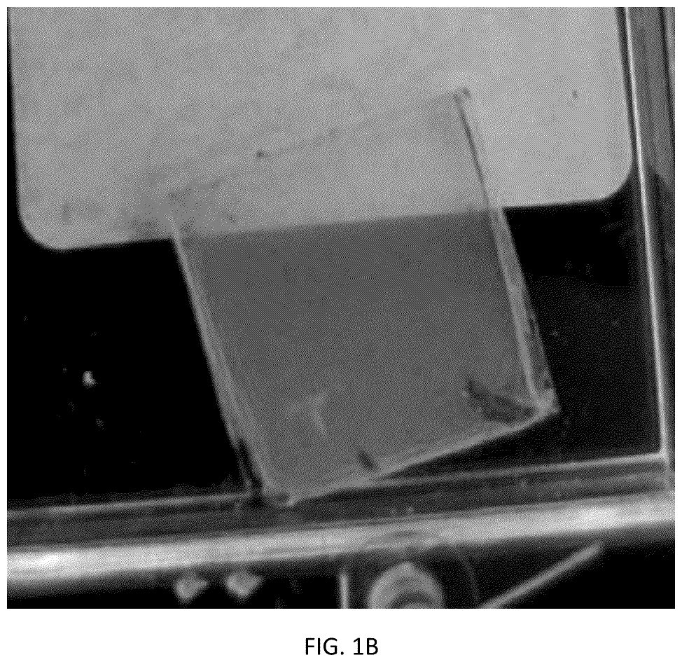

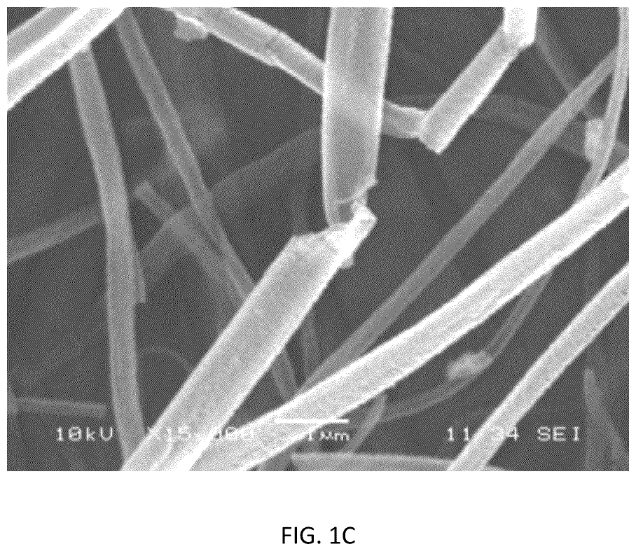

Referring now to FIGS. 1 -21 of the drawings, FIG. 1A shows a microscope slide with cellulose acetate prepared by electrospinning, FIG. 1B shows the cellulose acetate on a microscope slide after ZnO deposition and heating in air to remove the polymer template, and FIG. 1C shows a micrograph of typical nanoshells as observed in the SEM.

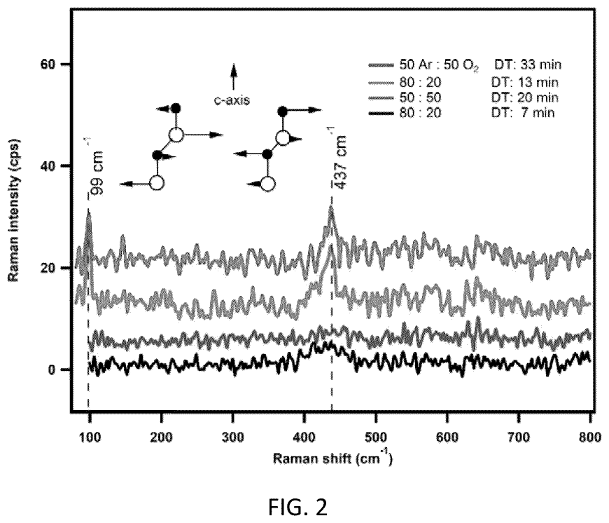

FIG. 2 is Raman spectra of ZnO nanoshells after heating in air. The samples were prepared at two different gas compositions and different deposition times. Two characteristic peaks corresponding to the excitation in the oxygen and zinc sub-lattices are observed.

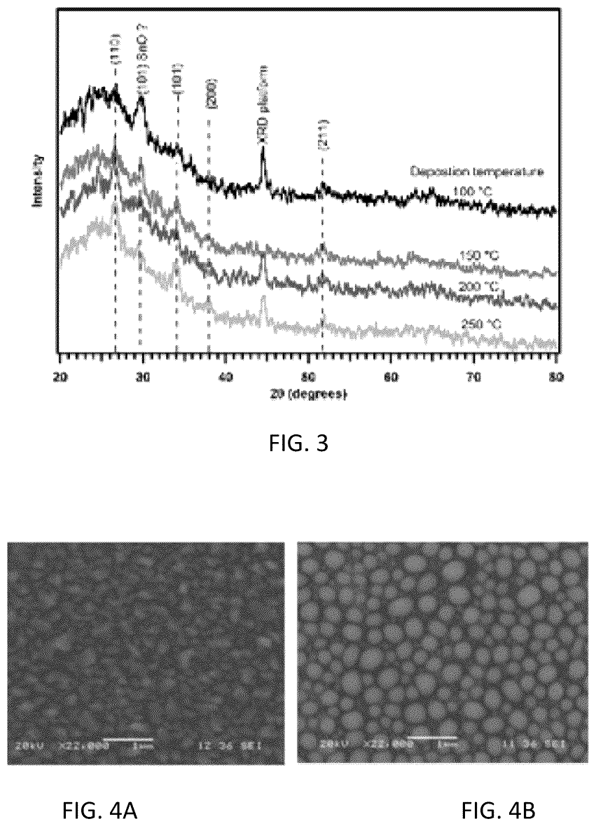

FIG. 3 is an X-ray Powder Diffraction (XRD) depiction whereby crystalline substances act as three-dimensional diffraction gratings for X-ray wavelengths similar to the spacing of planes in a crystal lattice and may be used in reviewing crystal structures and atomic spacing.

FIGS. 4A and 4B show the morphology of the tin semispherical particles deposited on glass, corresponding to substrate temperatures of 100° C. and 250° C., respectively.

Several mechanisms of nucleation and growth are explored in the synthesis of tin oxide nanostructures where tin semispherical particles are deposited by magnetron sputtering followed by thermal heating in air. An XRD of the tin semispherical particles is shown in FIG. 3 for different substrate temperatures. Variations in crystalline structure, SnO and SnO₂, and morphology can be modified by controlling the deposition conditions as shown. Increasing the temperature over the tin melting point Tₘ=231.9 C results in semi spherical particles as discovered in 1990, but in the present invention, these particles have been prepared at temperatures well below Tₘ (not shown). This methodology provides the opportunity of improving the nano-microstructure resulting in better sensing properties.

FIG. 5 is SERS signal of Rhodamine 6G for different silver films on glass substrates. Surface-enhanced Raman scattering (SERS) is a surface-sensitive technique that enhances Raman scattering by molecules adsorbed on rough metal surfaces or by nanostructures such as plasmonic-magnetic silica nanotubes wherein the enhancement factor can be as much as 10¹⁰ to 10¹¹. The spectra in the inset are the signals with the lowest amplification. Rhodamine 6G was used as the target molecule to be detected. The samples were deposited on glass at different sputtering conditions to produce different morphologies. The signal 510 of the test molecule on the bare glass substrate is shown in the inset 502 of FIG. 5. An increase in Raman signal was observed.

FIG. 6 is a chart showing sensitivity of ZnO thin film prepared at 50:50 02:Ar and FIG. 7 is a table of responses. Magnetron sputtering is used for the deposition of the ZnO metal oxide on top of electrospun polymer fibers that act as a template shaping the morphology of the material being deposited. Heating the samples in air allows removal of the polymeric template and the ZnO nanoshells are observed, as shown in FIG. 6. Raman spectroscopy measurements are shown in FIG. 7 of four samples prepared under different conditions of oxygen partial pressure and deposition time. The observed peaks are consistent with the formation of a wurtzite phase with 002 texture. Interestingly, the sample prepared with a smaller partial pressure of oxygen in the sputtering gas (20%), 70 nm of estimated thickness, shows Raman peaks comparable to a thicker sample, 140 nm of estimated thickness, prepared at a higher partial pressure of oxygen (50%). In a zinc oxide sensor, a morphological structure for a ZnO nanoshell uses a combination of electrospinning and magnetron sputtering deposition.

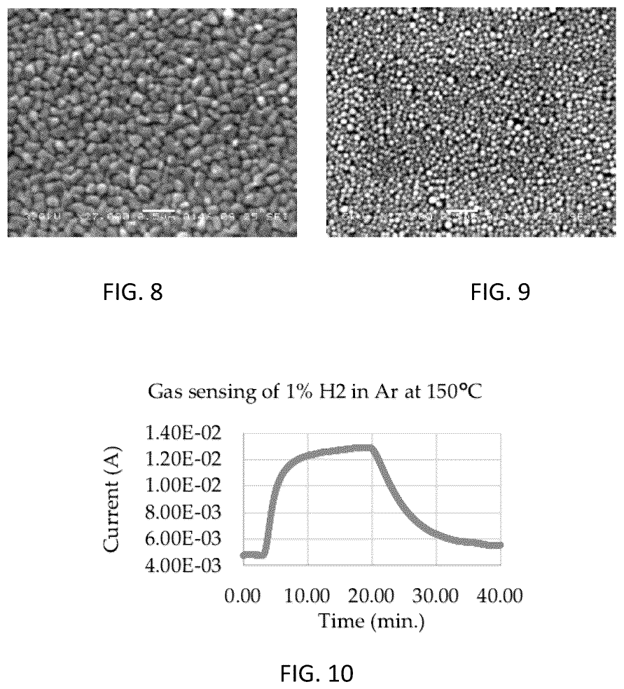

FIG. 8 is a chart showing morphology of a TONs (tin oxide nanoparticles) structure whereby SnO2 core-shell nanoparticles were prepared by de magnetron sputtering of tin nanoparticles directly on the IDTs, followed by heating in air. FIG. 9 is a chart showing morphology of another TONs structure and FIG. 10 shows the sensor response. Controlling the plasma processing parameters is possible to change the morphology of the core-shell TONs, as shown, to prepare semispherical nanoparticles by nucleation under melting temperature suppression. Initial testing of the gas response toward 1% hydrogen in argon is also shown.

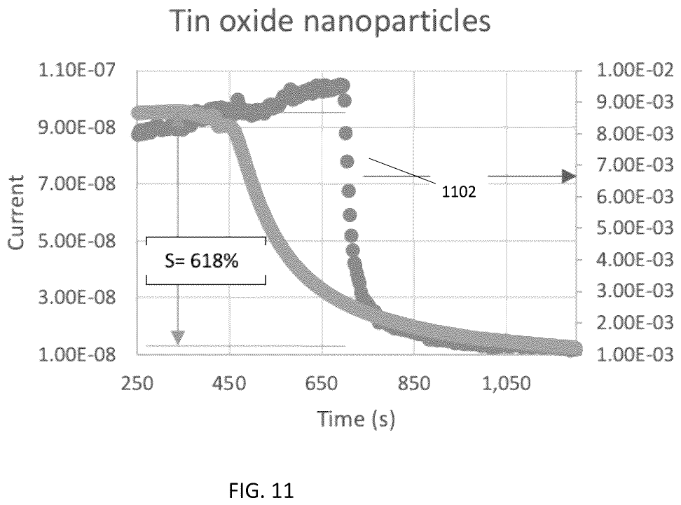

FIG. 11 is a chart showing a tin oxide nanoparticle plot of current versus time for two samples: sample 1102 had a heat post-treatment with the scale shown on the right, wherein S=100*(8.69−1.21)/1.21 and wherein S=618%. SO parts per million NO2 in N2.

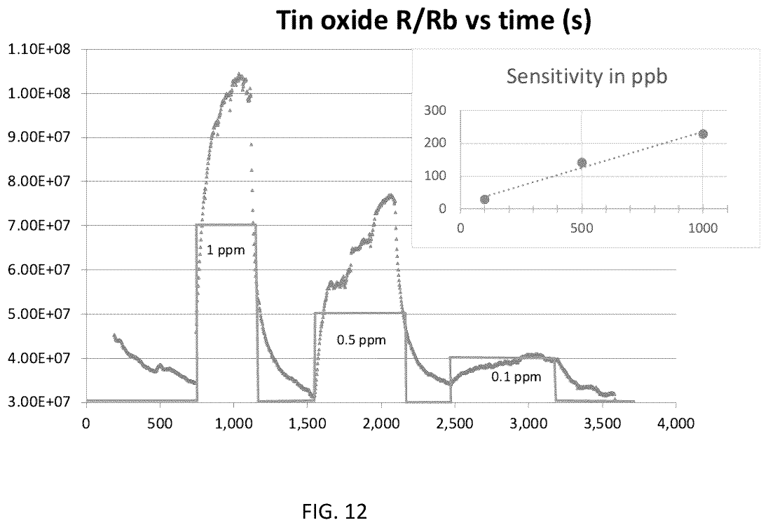

FIG. 12 is a chart showing relative resistance (R/Rb) of tin oxide sample for three different NO2 concentrations. The red line is a visual aid representing the introduction of the NO2 gas, at different concentrations, while maintaining the total gas flow constant. The insert shows the sensitivity as a function of concentration, in ppb where 1 ppm=1,000 ppb.

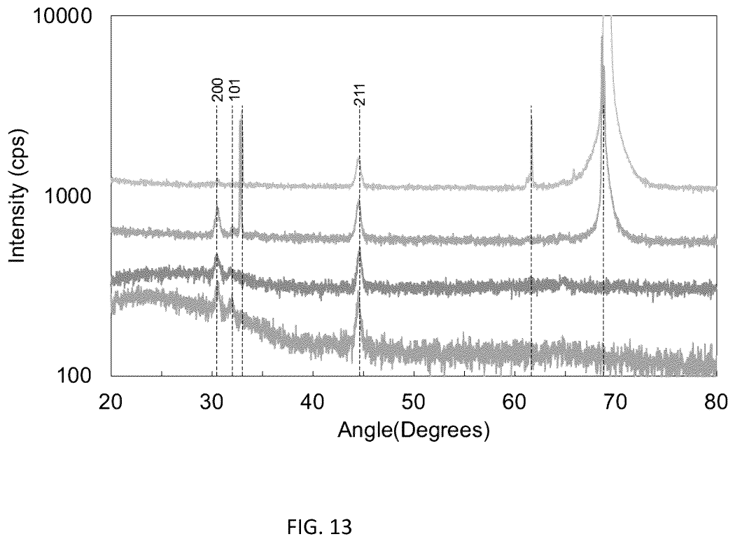

FIG. 13 is a chart showing XRD of Glass and SiOx with a deposition time of 30 seconds and deposition temperatures before thermal oxidation.

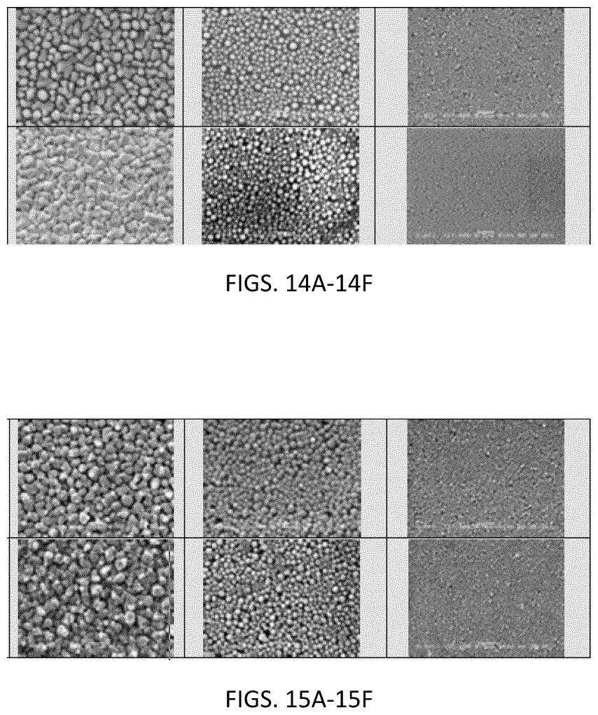

FIGS. 14A-14F show SEM images of the Sn nanoparticles at a 60, 30 and 15 second deposition duration at 200° C., using SiOx (top photos) and Glass (bottom photos) and FIGS. 15A-15F show SEM images of the SnO2 nanoparticles at a 60, 30 and 15 second deposition duration at 200° C., using SiOx (top photos) and Glass (bottom photos).

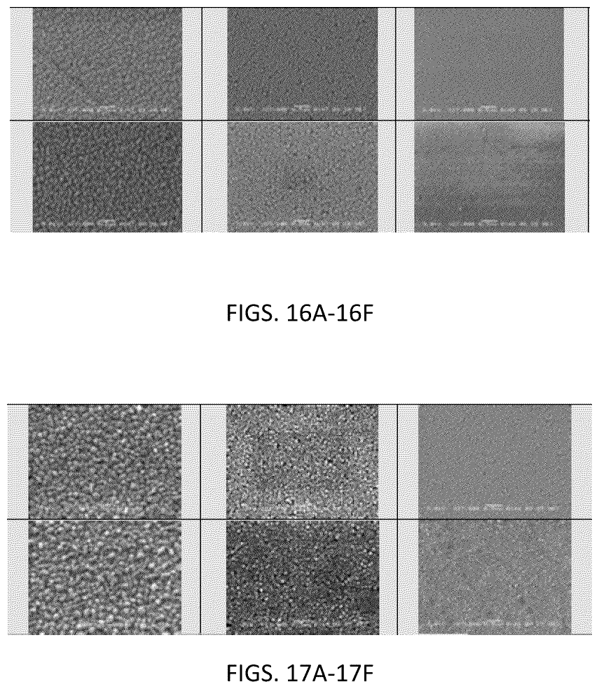

FIGS. 16A-16F show SEM images of the Sn nanoparticles at a 30, 15 and 10 second deposition duration at 150° C., using SiOx (top photos) and Glass (bottom photos) and FIGS. 17A-17F show SEM images of the SnO2 nanoparticles at a 30, 15 and 10 second deposition duration at 150° C., using SiOx (top photos) and Glass (bottom photos).

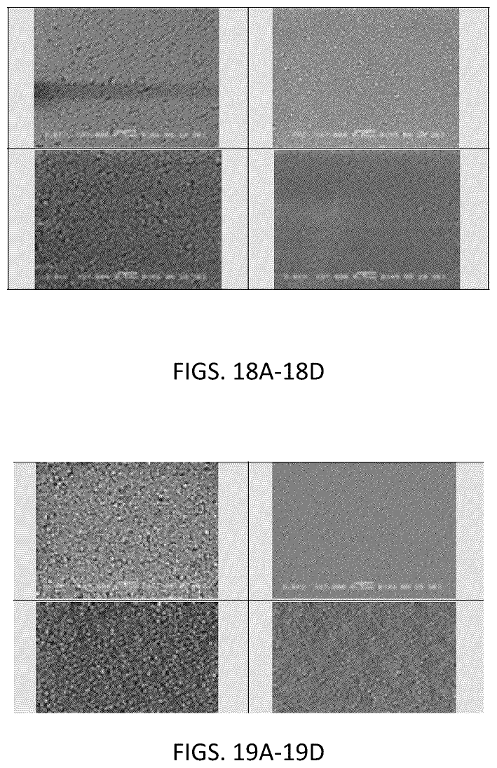

FIGS. 18A-18D show SEM images of the Sn nanoparticles at a 15 and 10 second deposition duration at 100° C., using SiOx (top photos) and Glass (bottom photos) and FIGS. 19A-19D show SEM images of the SnO2 nanoparticles at a 15 and 10 second deposition duration at 100° C., using SiOx (top photos) and Glass (bottom photos).

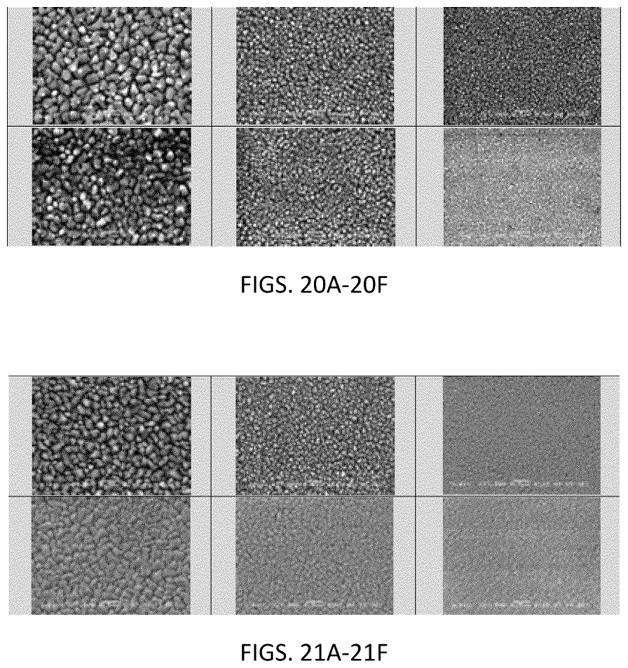

FIGS. 20A-20F show SEM images of the Sn nanoparticles at a 30, 15 and 10 second deposition duration at room temperature, using SiOx (top photos) and Glass (bottom photos) and FIGS. 21A-21F show SEM images of the SnO2 nanoparticles at a 30, 15 and 10 second deposition duration at room temperature, using SiOx (top photos) and Glass (bottom photos).

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A gas sensor for detecting a gas, the gas sensor comprising:
a polymer fiber substrate;
a metal oxide nanoshell disposed on the polymer substrate;
wherein the metal oxide nanoshell is deposited on the polymer fiber substrate by magnetron sputtering deposition.

2. The gas sensor according to claim 1 wherein the metal oxide is tin oxide.

3. The gas sensor according to claim 2 wherein the gas sensor is for detecting NO₂.

4. The gas sensor according to claim 3 wherein the polymer fiber substrate is a cellulose acetate substrate.

5. The gas sensor according to claim 3 wherein the polymer fiber substrate is prepared by electrospinning.

6. The gas sensor according to claim 1 wherein the metal oxide is zinc oxide.

7. The gas sensor according to claim 6 wherein the gas sensor is for detecting NH₃.

8. The gas sensor according to claim 6 wherein the polymer fiber substrate is a cellulose acetate substrate.

9. The gas sensor according to claim 6 wherein the polymer fiber substrate is prepared by electrospinning.

10. A method for making a gas sensor, the method comprising:
electrospinning polymeric fibers;
depositing a metal oxide on the polymeric fibers wherein a metal oxide nanoshell is produced on the polymeric fibers; and
heating the polymer fibers and metal oxide in air to remove the polymeric fibers;
wherein the gas sensor uses the nanoshell for a transduction process; and
wherein magnetron sputtering deposition is used for fabrication of the gas sensor.
